(19) United States
(12) Patent Application Publication
(10) Pub. No.: US 2026/0174761 A1
(43) Pub. Date: Jun. 25, 2026

(54) TRANSLOCATOR PROTEIN LIGANDS AND INFLAMMATORY DISEASES

(71) Applicant: TECHNION RESEARCH & DEVELOPMENT FOUNDATION LIMITED, Haifa (IL)

(72) Inventor: Moshe GAVISH, Haifa (IL)

(21) Appl. No.: 19/123,154

(22) PCT Filed: Nov. 2, 2023
(86) PCT No.: PCT/IL2023/051128

§ 371 Date: Apr. 22, 2025

Related U.S. Application Data
(60) Provisional application No. 63/421,667, filed on Nov. 2, 2022.

(51) Int. Cl. A61K 31/517; A61K 9/00; A61K 31/573; A61K 31/606; A61K 31/655; A61P 37/06

(52) U.S. Cl. CPC A61K 31/517; A61K 9/0019; A61K 31/573; A61K 31/606; A61K 31/655; A61P 37/06

(57) ABSTRACT

Methods for treating or reducing inflammation, in is in a site or an organ excluding the central nervous system of a subject in need thereof, by administering a pharmaceutical composition comprising the translocator protein ligand 2-Cl-MGV-1, are provided.

### CROSS-REFERENCE TO RELATED APPLICATIONS

This application claims the benefit of priority of U.S. Provisional Patent Application No. 63/421,667, titled “TRANSLOCATOR PROTEIN LIGANDS AND INFLAMMATORY DISEASES”, filed 2 Nov. 2022, the contents of which are incorporated herein by reference in their entirety.

### FIELD OF INVENTION

The present invention is in the field of translocator protein ligands and inflammation.

### BACKGROUND

The 18 kDa Translocator Protein (TSPO) is a mitochondrial protein that is primarily situated at contact sites between inner and outer mitochondrial membranes and is part of the mitochondrial permeability transition pore (MPTP). TSPO is involved in various cellular functions such as steroidogenesis, apoptosis, differentiation of neuronal progenitor cells, cholesterol transport, mitochondrial respiration, mitochondrial permeability transition pore opening, cellular proliferation, reactive oxygen species (ROS) generation, cell nuclear gene expression, and microglial and macrophage activation.

In recent studies, the current inventors discovered TSPO ligands, by expanding a quinazoline scaffold. The TSPO ligand, 2-chlorophenyl quinazolin-4-yl dimethylcarbamate (2-Cl-MGV-1), is a tricyclic compound, that was found by the inventors to inhibit lipopolysaccharide (LPS)-induced microglial activation. In contrast to classical TSPO ligands (e.g., PK 11195), that typically show pronounced lethal effects at high concentrations (above 100 μM), 2-Cl-MGV-1 has an advantage of showing very few lethal effects, even at high concentrations, while still protecting against neuronal damage.

Pharmaceutical compositions including compounds such as 2-Cl-MGV-1, and methods of using these compounds for the prevention and treatment of brain damage resulting from brain injury, and especially secondary brain damage due to traumatic brain injury (TBI), have been disclosed. Nonetheless, these compounds therapeutic potential in peripheral inflammatory diseases, outside the CNS, has not been addressed so far.

Inflammatory bowel disease (IBD) is a group of inflammatory conditions of the colon and small intestine, comprising Crohn's disease and ulcerative colitis. Treatment options that ameliorate IBD symptoms, and prolong the remission periods, comprise antibiotics, (e.g. metronidazole and ciprofloxacin), amino salicylic acid (ASA) anti-inflammatory drugs, steroids, (e.g. prednisone, prednisolone or budesonide), immunomodulators (e.g. azathioprine and methotrexate), and biologic treatments, based on monoclonal antibodies directed against specific molecules with immunomodulatory or anti-inflammatory activity (e.g. anti-TNF-α antibody).

Macular degeneration, also known as age-related macular degeneration (AMD or ARMD), is a disease which may result in blurred or no vision in the center of the visual field. There are two patterns of AMD: dry and wet. The dry form of AMD is characterized by drusen cellular debris, accumulated in the macula that gradually damages light-sensitive cells and leads to vision loss. In the wet form of AMD, blood vessels grow under the macula, causing blood leakage into the retina. While anti-vascular endothelial cell growth factor (VEGF) therapy may postpone AMD aggravation in the case of wet AMD, no treatment options are available for dry AMD.

There is a great need for therapies that may reduce or inhibit peripheral inflammation, such as, but not limited to, IBD and AMD.

### SUMMARY

According to a first aspect, there is provided a method for treating or reducing peripheral inflammation, or a symptom associated therewith in a subject in need thereof, wherein the peripheral inflammation is in a site or an organ excluding the central nervous system (CNS) of the subject, the method comprising administering to the subject a therapeutically effective amount of a pharmaceutical composition comprising the compound represented by Formula I, wherein R1 and R2 are each independently a linear or branched C1-C12 alkyl; R3 is a halogen; and n is 1, 2, 3, 4 or 5; including salts thereof, thereby treating or reducing peripheral inflammation or a symptom associated therewith in a subject in need thereof.

According to another aspect, there is provided a pharmaceutical composition comprising the compound represented by Formula I, for use in treatment or reduction of peripheral inflammation, or a symptom associated therewith, in a subject in need thereof, wherein the peripheral inflammation is in a site or an organ excluding the CNS of the subject.

In some embodiments, the compound represented by Formula I comprises wherein said compound comprises 2-Cl-MGV-1.

In some embodiments, the site or organ is selected from a group consisting of: intestine, eye, lung, heart, and any combination thereof, of the subject.

In some embodiments, the subject is afflicted with a disease selected from the group consisting of: an autoimmune disease, an age-related disease, a pathogen-induced infectious disease, and any combination thereof.

In some embodiments, the autoimmune disease is inflammatory bowel disease (IBD).

In some embodiments, the treating or reducing peripheral inflammation comprises reducing colon shortening in the subject.

In some embodiments, the age-related disease is age-related macular degeneration (AMD).

T In some embodiments, the treating or reducing peripheral inflammation comprises reducing the expression, secretion, or both, of a pro-inflammatory modulator, in the subject.

In some embodiments, the pro-inflammatory modulator is selected from the group consisting of: interleukin 1β (IL-1β), IL-6, tumor necrosis factor α (TNF-α), interferon γ (IFN-γ), reactive oxidative species (ROS), IL-8, complement C3, complement C5, and any combination thereof.

In some embodiments, the administering comprises orally administering, intravenously administering, intramuscularly administering, or any combination thereof.

In some embodiments, the method further comprises administering to the subject an anti-inflammatory agent.

In some embodiments, the pharmaceutical composition for use is formulated for administration via a route selected from the group consisting of: oral administration, intravenous administration, intramuscular administration, and any combination thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 includes a bar graph showing that interleukin (IL) 6 levels in the gut of IBD mice are reduced in response to 2-CL-MGV-1 treatment. Nine-weeks-old C57BL6 male mice were given 2.5% dextran sulfate sodium (DSS) in drinking water for 5 days. Naive mice were given water only. The treatments: 2-CL-MGV-1 and dexamethasone were given on day 4 for four days. DSS, DSS treated mice without treatment; DSS+2-CL-MGV-1, DSS and 15 mg/kg 2-CL-MGV-1; DEX, DSS and 0.75 mg/kg dexamethasone. ANOVA followed by Bonferroni's post-hoc test was performed. ***p<0.00001 compared to control.

FIG. 2 includes a bar graph showing that the treatment with 2-CL-MGV-1 reduces the shortening of IBD mice colon. Nine-weeks-old C57BL6 male mice were given 2.5% DSS in drinking water for 5 days. Naive mice were given water only. The treatments: 2-CL-MGV-1 and dexamethasone were given on day 4 for four days. DSS, DSS treated mice without treatment; DSS+2-CL-MGV-1, DSS and 15 mg/kg 2-CL-MGV-1; DEX, DSS and 0.75 mg/kg dexamethasone.

FIGS. 3A-3D include vertical bar graphs demonstrating the in vitro effect of 2-CL-MGV-1 on the levels of reactive oxygen species (ROS; 3A), IL-8 (3B), complement C3 (3C) and complement C5 (3D), that are secreted from human retinal pigment epithelium (RPE)-19 cell line stimulated with tumor necrosis factor alpha (TNF-α) and interferon gamma (INF-γ). TNF-α and INF-γ stimulation (TI).

FIG. 4 includes a vertical bar graph showing that IL 6 levels in the gut of IBD mice are reduced in response to 2-CL-MGV-1 treatment. C57BL female mice aged of 8-10 weeks were given 2.5% DSS in their drinking water for 7 days. Vehicle group mice were given water only and DMSO injections on the 4th day for 4 days. DSS group received DSS with DMSO (as a vehicle) that were given on the 4th day for 4 days. The treatment group received 2-CL-MGV-1 (15 mg/kg) and DMSO as a vehicle were given on the 4th day for 4 days. There was 64% decrease in IL-6 content in the colon of mice treated with 2-CL-MGV-1 compared to mice treated with DMSO (DSS+ vehicle). ANOVA followed by Bonferroni's post-hoc test was performed. ***p<0.001 compared to control.

FIG. 5 includes a vertical bar graph showing that the treatment with 2-CL-MGV-1 reduces the shortening of IBD mice colon. C57BL female mice aged of 8-10 weeks were given 2.5% DSS in their drinking water for 7 days. Vehicle group mice were given water only and DMSO injections on the 4th day for 4 days. DSS group received DSS with DMSO (as a vehicle) that were given on the 4th day for 4 days. The treatment group received 2-CL-MGV-1 (15 mg/kg) and DMSO (as a vehicle) that were given on the 4th day for 4 days. There was a 26% increase in colon length in mice treated with 2-CL-MGV-1 to a level of the vehicle group in compared to mice treated only in DMSO (DSS+ vehicle). ANOVA followed by Bonferroni's post-hoc test was performed. ***p<0.001 compared to control.

### DETAILED DESCRIPTION

The present invention, in some embodiments, is based, at least partially, on the surprising finding, that a mitochondrial translocator protein (TSPO) ligand, namely, 2-chlorophenyl quinazolin-4-yl dimethylcarbamate (2-Cl-MGV-1), had significantly reduced secretion of a pro-inflammatory cytokine, including IL-6, in a murine model of peripheral inflammation, i.e., outside the central nervous system (CNS), specifically, an inflammatory bowel disease (IBD) model. It is important to note that the inventors found that the effect was about 75% more pronounced compared to treatment with the corticosteroid dexamethasone (DEX), which is known to be used in the treatment of IBD. In some embodiments, treatment of IBD mice with 2-Cl-MGV-1 reduced colon shortening, a pathologic hallmark of IBD, and this effect was also augmented compared to DEX treatment. In some embodiments, the present invention is further based, at least in part, on the surprising finding that 2-Cl-MGV-1 reduced inflammation in an in vitro model of age-related macular degeneration (AMD).

The present invention, in some embodiments, provides a method for treating or reducing peripheral inflammation or a symptom associated therewith in a subject in need thereof.

As used herein, the term “symptom associated therewith” refers to any symptom that accompanies the inflammation, derived from the inflammation, causes the inflammation (or one of the causes), or any combination thereof. In some embodiments, the method disclosed herein is for treating acute inflammation (e.g., inflammation as a response to acute injury or pathogen-induced infection). In some embodiments, a symptom associated with acute inflammation comprises flushed skin at the site of the injury, pain, swelling, heat, or any combination thereof. In some embodiments, the method disclosed herein is for treating a chronic inflammation (e.g., inflammation in an autoimmune disease). In some embodiments, a symptom associated with chronic inflammation comprises abdominal pain, chest pain, fatigue, fever, joint pain or stiffness, mouth sores, skin rash, or any combination thereof.

In some embodiments, the method comprises administering to the subject a therapeutically effective amount of a pharmaceutical composition comprising a compound represented by the structure of formula I:

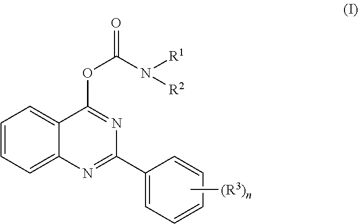

wherein R1 and R2 are each independently a linear or branched C1-C12 alkyl; R3 is a halogen; and n is 1, 2, 3, 4 or 5; including salts thereof.

In some embodiments, the pharmaceutical composition comprises a compound according to formula I, wherein R¹ and R² are each independently selected from: methyl, ethyl, propyl, isopropyl, n-butyl, sec-butyl, isobutyl, tert-butyl, or any combination thereof.

In some embodiments, the pharmaceutical composition comprises a compound according to formula I, wherein R¹ is different from R².

In some embodiments, the pharmaceutical composition comprises a compound according to formula I, wherein n is 1 or 2.

In some embodiments, the pharmaceutical composition comprises a compound according to formula I, wherein n R³ is Cl, Br or F or a combination thereof.

In some embodiments, the pharmaceutical composition comprises a compound selected from:

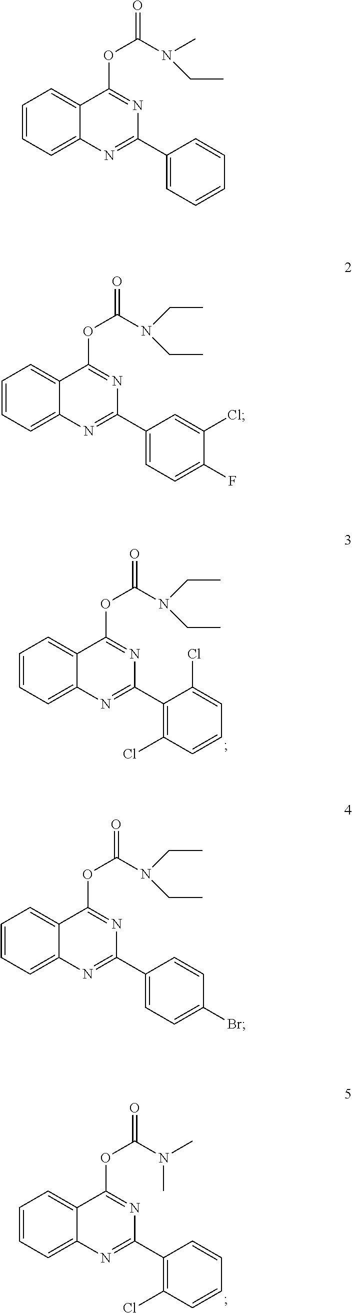 [SMILES: CCN(C)C(=O)Oc1nc(-c2ccccc2)nc2ccccc12 ; CCN(CC)C(=O)Oc1nc(-c2ccc(F)c(Cl)c2)nc2ccccc12 ; CCN(CC)C(=O)Oc1nc(-c2c(Cl)cccc2Cl)nc2ccccc12 ; CCN(CC)C(=O)Oc1nc(-c2ccc(Br)cc2)nc2ccccc12 ; CN(C)C(=O)Oc1nc(-c2ccccc2Cl)nc2ccccc12]

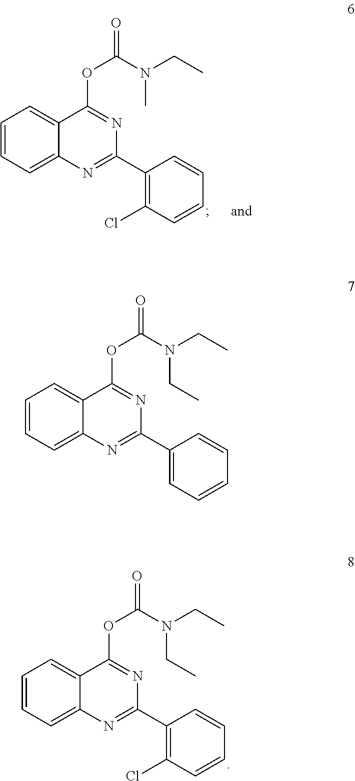 [SMILES: CCN(C)C(=O)Oc1nc(-c2ccccc2Cl)nc2ccccc12 ; CCN(CC)C(=O)Oc1nc(-c2ccccc2)nc2ccccc12 ; CCN(CC)C(=O)Oc1nc(-c2ccccc2Cl)nc2ccccc12]

In some embodiments, the pharmaceutical composition comprises a compound according to formula I, wherein R3 is Cl.

In some embodiments, the pharmaceutical composition comprises a compound which is represented by the structure of Formula 5, as disclosed herein.

In some embodiments, the pharmaceutical composition comprises 2-chlorophenyl quinazolin-4-yl dimethylcarbamate (2-Cl-MGV-1), represented by Formula 5.

In some embodiments, the method comprises administering to a subject a therapeutically effective amount of a pharmaceutical composition comprising 2-Cl-MGV-1.

In some embodiments, a peripheral inflammation comprises any inflammation in a site or organ excluding the central nervous system (CNS) of a subject. In some embodiments, peripheral inflammation excludes inflammation in or of the CNS of a subject. In some embodiments, a peripheral inflammation comprises any inflammation in a peripheral site or organ.

As used herein, the terms “organ” or “site” encompass any site or organ in the body of a subject, excluding the CNS of the subject. As used herein, the terms “peripheral organ” and “organ”, and the terms “peripheral site” and “site”, are used interchangeably. In some embodiments, peripheral inflammation comprises activation of any one of the innate or adaptive immune systems, and both, of a subject. In some embodiments, activation of an immune response, as disclosed herein, comprises expression, secretion/release, or both, of at least one pro-inflammatory cytokine, in, from, or both, a site or an organ excluding the CNS of a subject. In some embodiments, the peripheral inflammation is in a site or an organ excluding the CNS and the heart of a subject. In some embodiments, the peripheral inflammation is in a site or an organ excluding the CNS and the cardiovascular system.

In some embodiments, the subject is afflicted with a peripheral inflammatory disease. In some embodiments, peripheral inflammatory disease or peripheral inflammation, comprises increased expression, secretion, or both, of at least one pro-inflammatory modulator in a peripheral site or organ of a subject. As used herein the term “pro-inflammatory modulator” refers to any molecule that is known to be capable of inducing or activating a pro-inflammatory process, to be derived from a pro-inflammatory process, to be secreted by a pro-inflammatory cell, or any combination thereof. In some embodiments, a pro-inflammatory modulator comprises a protein or a small molecule. In some embodiments, a pro-inflammatory modulator is selected from: a cytokine, a chemokine, reactive oxygen species (ROS), a complement system protein, or any combination thereof. In some embodiments, a pro-inflammatory modulator comprises a pro-inflammatory cytokine. In some embodiments, a pro-inflammatory cytokine comprises interleukin (IL)-1, IL-2, IL-5, IL-6, IL-17, interferon (IFN)-γ, tumor necrosis factor (TNF)-α, or any combination thereof. In some embodiments, a pro-inflammatory modulator comprises a pro-inflammatory chemokine. In some embodiments, a pro-inflammatory chemokine comprises IL-8. In some embodiments, a pro-inflammatory modulator comprises reactive oxygen species (ROS). As used herein, the term “ROS” refers to highly reactive chemicals formed from O₂. Examples of ROS include, but not limited to: peroxide, superoxide, hydroxyl radical, singlet oxygen, alpha-oxygen, or any combination thereof. In some embodiments, a pro-inflammatory modulator comprises a complement system protein. In some embodiments, a complement system protein comprises protein C3, protein C5, or both. In some embodiments, the level of at least one pro-inflammatory modulator is above a predetermined threshold. In some embodiments, the subject is characterized by increased level of at least one pro-inflammatory modulator, in a peripheral site or organ, compared to a negative control subject. As used herein, the term “negative control subject” refers to a subject that is not afflicted with peripheral inflammation. In some embodiments, a negative control subject comprises a healthy subject. In some embodiments, the subject is characterized by increased level of at least one pro-inflammatory modulator in a peripheral site or an organ, compared to a healthy subject. In some embodiments, a peripheral site or an organ comprises the affected inflamed site or organ. In some embodiments, a peripheral site or organ comprises the serum of a subject. In some embodiments, the method further comprising a preceding step, comprising determining the level of at least one pro-inflammatory modulator, comprising a cytokine, a chemokine, ROS, a complement system protein, or any combination thereof, in a peripheral site or organ of a subject. In some embodiments, the preceding step comprises a selection step of a subject to be treated with the method disclosed herein, by determining the level of at least one pro-inflammatory modulator in a sample obtained or derived from the subject. In some embodiments, the sample comprises serum of a subject. In some embodiments, the sample comprises a biopsy from a peripheral inflamed organ, tissue, cell, or any combination thereof. In some embodiments, elevated level of at least one pro-inflammatory modulator is within the subject's serum. In some embodiments, elevated level of at least one pro-inflammatory modulator is within the affected inflamed organ, tissue, cell, or any combination thereof. In some embodiments, elevated level of at least one pro-inflammatory modulator level comprises protein expression level. In some embodiments, elevated level of at least one pro-inflammatory modulator comprises mRNA expression level. In some embodiments, elevated level of at least one pro-inflammatory modulator is by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 200%, at least 500%, or any combination thereof. Each possibility presents a separate embodiment of the invention.

In some embodiments, a peripheral inflammatory disease comprises increased level of at least one pro-inflammatory cytokine in a peripheral site or organ of a subject. In some embodiments, the level of at least one pro-inflammatory cytokine is above a pre-determined threshold. In some embodiments, the subject is characterized by increased level of at least one pro-inflammatory cytokine, in a peripheral site or organ, compared to a negative control subject. In some embodiments, a negative control subject comprises a healthy subject. In some embodiments, the subject is characterized by increased level of at least one pro-inflammatory cytokine, in a peripheral site or an organ, compared to a healthy subject. In some embodiments, a pro-inflammatory cytokine comprises interleukin (IL)-1, IL-2, IL-5, IL-6, IL-17, interferon (IFN)-γ, tumor necrosis factor (TNF)-α, or any combination thereof. In some embodiments, the method further comprising a preceding step, comprising determining the level of at least one pro-inflammatory cytokine in a peripheral site or organ of a subject. In some embodiments, the preceding step comprises a selection step of a subject to be treated with the method disclosed herein, by determining the level of at least one pro-inflammatory cytokine in a sample obtained or derived from the subject. In some embodiments, the sample comprises serum of a subject. In some embodiments, the sample comprises a biopsy from a peripheral inflamed organ, tissue, cell, or any combination thereof. In some embodiments, elevated level of at least one pro-inflammatory cytokine is within the subject's serum. In some embodiments, elevated level of at least one pro-inflammatory cytokine is within the affected inflamed organ, tissue, cell, or any combination thereof. In some embodiments, elevated level of at least one pro-inflammatory cytokine level comprises protein expression level. In some embodiments, elevated level of at least one pro-inflammatory cytokine level comprises mRNA expression level. In some embodiments, elevated level of at least one pro-inflammatory cytokine is by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 200%, at least 500%, or any combination thereof. Each possibility presents a separate embodiment of the invention.

In some embodiments, a peripheral inflammatory disease comprises at least one pro-inflammatory characteristic or immune system cell response selected from: increased T helper 1 (Th1) cell response, increased Th17 response, reduced Th2 response, reduced T regulatory cell (Treg) response, increased MI macrophage response, reduced M2 macrophage response, increased B effector (Beff) cell response, reduced B regulatory (Breg) response, increased complement activation, or any combination thereof, in a peripheral site or an organ of a subject, compared to a negative control subject, as disclosed herein. In some embodiments, a negative control subject comprises a subject that is not afflicted with peripheral inflammation. In some embodiments, a negative control subject comprises a healthy subject. As used herein, the term “an immune system cell response” refers to at least one of: immune system cell activation, immune system cell proliferation, a cytokine expression within the immune system cell, a cytokine secretion from the immune system cell, or any combination thereof. A known skilled in the art would know how to examine the specific immune system cell response. In this manner, non-limiting example for increased Th17 response may be enhanced proliferation of Th17 cells, or enhanced expression, secretion, or both, of IL-17, compared to healthy subject. Non-limiting example for increased complement activation may be increased level of C3 protein, or C5 protein, compared to healthy subject. An additional example for reduced Treg response, may be reduced levels of CD4⁺CD25⁺Foxp3⁺ cells, compared to healthy subject, or reduced suppressor activity of Treg that can be examined by proliferation of responder T cells, incubated in the presence of Tregs.

In some embodiments, reducing peripheral inflammation, by the method disclosed herein, comprises at least one of: reducing the level of a pro-inflammatory cytokine, increasing the level of an anti-inflammatory cytokine, reducing Th1 response, reducing Th17 response, increasing Th2 response, increasing Treg response, reducing MI macrophage response, increasing M2 macrophage response, reducing Beff response, increasing Breg response, reducing complement activation, or any combination thereof. In some embodiments, reducing peripheral inflammation comprises reducing expression, secretion, or both, of at least one pro-inflammatory modulator. In some embodiments, a pro-inflammatory modulator comprises a protein or a small molecule. In some embodiments, a pro-inflammatory modulator is selected from: a cytokine, a chemokine, ROS, a complement system protein, or any combination thereof. In some embodiments, reducing peripheral inflammation comprises reducing expression, secretion, or both of a pro-inflammatory cytokine encoding gene, transcript transcribed therefrom, a protein product thereof, or any combination thereof. In some embodiments, a pro-inflammatory cytokine is selected from: IL-1, IL-2, IL-5, IL-6, IL-17, IFN-γ, TNF-α, or any combination thereof. In some embodiments, the method further comprising a following step of determining the level of a pro-inflammatory modulator in the subject treated with a pharmaceutical composition comprising a compound according to formula I, as disclosed herein. In some embodiments, evaluation of treatment efficacy comprises determining the level of a pro-inflammatory modulator in a peripheral site or organ of a subject after treating with the method disclosed herein. In some embodiments, the level of a pro-inflammatory modulator is determined in a sample comprising a tissue, cell, biopsy, fragment, or any combination thereof, being derived from the inflamed organ of the subject. In some embodiments, the level of a pro-inflammatory modulator is determined within the subject's serum.

In some embodiments, the level of a pro-inflammatory modulator in the subject treated with a pharmaceutical composition comprising a compound according to formula I is compared to a positive control subject. In some embodiments, reducing the level of a pro-inflammatory modulator by the method disclosed herein, is compared to a predetermined threshold. In some embodiments, reducing the level of a pro-inflammatory modulator is compared to a positive control subject. As used herein, the term “positive control subject” comprises a subject afflicted with the same peripheral inflammatory disease or condition, as the subject disclosed herein, but not treated with a pharmaceutical composition comprising a compound according to formula I, as disclosed herein. In some embodiments, a positive control subject comprises a subject afflicted with the same peripheral inflammatory disease or condition, as the subject disclosed herein, but not treated with a pharmaceutical composition comprising 2-Cl-MGV-1. In some embodiments, a positive control subject comprises a subject afflicted with the same disease or condition as the subject disclosed herein, however treated with other immunomodulatory or anti-inflammatory agent, not comprising 2-Cl-MGV-1. In some embodiments, other immunomodulatory or anti-inflammatory agent comprises corticosteroid. In some embodiments, a corticosteroid comprises dexamethasone (DEX). In some embodiments, reducing peripheral inflammation, or reducing a pro-inflammatory modulator, is by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or any combination thereof. Each possibility presents a separate embodiment of the invention.

In some embodiments, peripheral inflammation is within a site or an organ selected from: intestine, eye, lung, heart, or any combination thereof, of the subject disclosed herein. In some embodiments, peripheral inflammation is within a site or an organ selected from: intestine, eye, lung, or any combination thereof, of the subject disclosed herein.

In some embodiments, the subject is afflicted with a disease being selected from: an autoimmune disease, an aging-related disease, a pathogen-induced infection disease, or any combination thereof.

In some embodiments, the method disclosed herein is for treating or reducing peripheral inflammation in a subject afflicted with an autoimmune disease. In some embodiments, a peripheral inflammation comprises an autoimmune disease that impairs an organ or a site excluding the CNS. In some embodiments, a peripheral inflammation comprises an autoimmune disease that develops in an organ excluding the CNS. In some embodiments, an autoimmune disease comprises a disease selected from: inflammatory bowel disease (IBD), type 1 diabetes, rheumatoid arthritis (RA), psoriasis/psoriatic arthritis, systemic lupus erythematosus (SLE), addison's disease, graves' disease, sjögren's syndrome, hashimoto's thyroiditis, myasthenia gravis, autoimmune vasculitis, pernicious anemia, celiac disease, scleroderma, or any combination thereof. In some embodiments, an autoimmune disease comprises IBD.

In some embodiments, the subject is afflicted with an age-related disease. As used herein, the term “age-related disease” refers to a disease that its distribution or frequency increases among older or elderly subjects. In some embodiments, age-related disease comprises a disease or condition manifested by increased levels of reactive oxidative species (ROS). In some embodiments, age-related disease comprises damaged mitochondria or mitochondrial dysfunction. As used herein, the term “ROS” refers to highly reactive chemicals formed from O₂. Examples of ROS include, but not limited to: peroxide, superoxide, hydroxyl radical, singlet oxygen, alpha-oxygen, or any combination thereof. In some embodiments, elevated ROS levels are present in the subject's serum. In some embodiments, elevated levels of ROS are present within the affected inflamed organ, tissue, cell, or any combination thereof. In some embodiments, elevated ROS levels comprise protein expression level. In some embodiments, elevated levels comprise mRNA expression level. In some embodiments, elevated ROS levels are above a predetermined threshold. In some embodiments, elevated ROS levels are compared to a negative control subject. As used herein the term “negative control subject” refers to a subject that is not afflicted with age-related disease. In some embodiments, negative control subject comprises a healthy subject. In some embodiments, negative control subject comprises a younger subject compared to the subject disclosed herein. In some embodiments, a negative control subject comprises a healthy subject of age within the range 20-50. In some embodiments, a negative control subject comprises a non-smoker healthy subject. In some embodiments, the method further comprising a preceding step, comprising determining the level of ROS in a peripheral site or organ of a subject. In some embodiments, the preceding step comprises a selection step of a subject to be treated with the method disclosed herein, by determining the level of ROS in a sample obtained or derived from the subject. In some embodiments, the sample comprises serum of a subject. In some embodiments, the sample comprises a biopsy from a peripheral inflamed organ, tissue, cell, or any combination thereof. In some embodiments, ROS levels are elevated by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 200%, at least 500%, or any combination thereof. Each possibility presents a separate embodiment of the invention. In some embodiments, elevated ROS levels are elevated by at least 40%. In some embodiments, elevated ROS levels are elevated by at least one of: 20%-100%, 30%-90%, 30%-80%, 40%-70%, and 40%-60%. Each possibility presents a separate embodiment of the invention.

In some embodiments, age-related disease comprises at least one of: age-related macular degeneration (AMD), refractive error, osteoarthritis, chronic obstructive pulmonary disease (COPD), type 2 diabetes, or any combination thereof. In some embodiments, refractive error is associated with AMD. In some embodiments, refractive error comprises a risk factor for AMD. In some embodiments, age-related disease comprises AMD.

In some embodiments, the method disclosed herein is for treating or reducing peripheral inflammation in a subject afflicted with a pathogen-induced infection disease. In some embodiments, the pathogen comprises bacteria. In some embodiments, the pathogen comprises a virus. In some embodiments, the pathogen comprises a fungus. In some embodiments, the infection comprises infection of at least one organ selected from: eye, intestine, heart, lung, or any combination thereof. In some embodiments, the subject is afflicted with pathogen-induced heart infection. In some embodiments, heart infection is selected from: endocarditis, myocarditis, pericarditis, or any combination thereof. In some embodiments, the subject is afflicted with pathogen-induced lung infection. In some embodiments, lung infection is selected from: pneumonia, influenza, COVID-19, tuberculosis (TB), bronchitis, or any combination thereof.

In some embodiments, the method disclosed herein is for treating cytokine storm or cytokine storm syndrome. As used herein, the terms “cytokine storm” and “hypercytokinemia” are used interchangeably. As used herein, cytokine storm refers to a physiological reaction in which the innate immune system causes an uncontrolled and excessive release of a pro-inflammatory cytokine. In some embodiments, cytokine storm can cause multisystem organ failure and death. In some embodiments, cytokine storm syndrome comprises a condition that results in a cytokine storm. Examples for cytokine storm syndrome include, but are not limited to: sepsis, familial hemophagocytic lymphohistiocytosis, Epstein-Barr virus-associated hemophagocytic lymphohistiocytosis, systemic or non-systemic juvenile idiopathic arthritis-associated macrophage activation syndrome, NLR Family CARD domain containing 4 (NLRC4) macrophage activation syndrome, cytokine release syndrome (CRS), or any combination thereof. As used herein, CRS refers to a form of systemic inflammatory response syndrome (SIRS) that can be triggered by an infection pathogen and/or a certain drug.

In some embodiments, the method disclosed herein is for treating IBD. In some embodiments, the method is for treating a symptom associated with IBD, comprising persistent diarrhea, abdominal pain, rectal bleeding, bloody stool, weight loss, fatigue, or any combination thereof. In some embodiments, the method is for treating at least one disease selected from: Crohn's disease, ulcerative colitis, ischemic colitis, diversion colitis, radiation enteritis, microscopic colitis (e.g., lymphocytic colitis or collagenous colitis), or any combination thereof. In some embodiments, treating or reducing peripheral inflammation comprises treating or reducing peripheral inflammation in IBD. In some embodiments, treating or reducing peripheral inflammation comprises reducing a pro-inflammatory cytokine expression and/or secretion. In some embodiments, reducing comprises reducing protein expression. In some embodiments, reducing comprises reducing mRNA expression. In some embodiments, a pro-inflammatory cytokine comprises IL-6. In some embodiments, reducing a pro-inflammatory cytokine is within the subject's intestine. As used herein, the terms: “intestine” and “colon” are used interchangeably. In some embodiments, reducing a pro-inflammatory expression is by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or any combination thereof. Each possibility presents a separate embodiment of the invention. In some embodiments, reducing a pro-inflammatory expression is within the range of 60%-90%. In some embodiments, reducing a pro-inflammatory expression is compared to a positive control subject, as disclosed herein. In some embodiments, a positive control subject comprises a subject afflicted with IBD, not treated with the pharmaceutical composition disclosed herein. In some embodiments, a positive control subject comprises a subject afflicted with IBD treated with other immunomodulatory or anti-inflammatory agent, comprising corticosteroid. In some embodiments, a corticosteroid comprises dexamethasone (DEX).

In the later stages of IBD the colon can narrow and shorten, impairing water absorption. In some embodiments, treating comprises reducing colon shortening in the subject. In some embodiments, reducing colon shortening is by at least one of: 2%, at least 5%, at least 10%, at least 15%, at least 20%, at least 25%, at least 35%, at least 40%, at least 45%, at least 50%, at least 55%, at least 60%, at least 65%, at least 70%, at least 80%, at least 85%, at least 90%, at least 95%, at least 99%, or any combination thereof. Each possibility presents a separate embodiment of the invention. In some embodiments, reducing colon shortening is compared to a positive control subject, as disclosed herein. In some embodiments, a positive control subject comprises a subject afflicted with IBD, not treated with the pharmaceutical composition disclosed herein. In some embodiments, a positive control subject comprises a subject afflicted with IBD treated with other immunomodulatory or anti-inflammatory agent, comprising corticosteroid. In some embodiments, a corticosteroid comprises dexamethasone (DEX).

In some embodiments, the method disclosed herein is for treating age-related macular degeneration (AMD). In some embodiments, the method is for treating AMD-associated symptom, comprising blurry vision, a dark empty area or blind spot in the center of vision, loss of central vision, or any combination thereof. In some embodiments, treating or reducing peripheral inflammation comprises treating or reducing peripheral inflammation in AMD. In some embodiments, treating AMD comprises reducing macular degeneration. In some embodiments, treating or reducing peripheral inflammation in AMD comprises reducing expression and/or secretion of a pro-inflammatory modulator selected from: a cytokine, a chemokine, ROS, complement system protein, or any combination thereof. In some embodiments, reducing comprises reducing protein expression. In some embodiments, reducing comprises reducing mRNA expression. In some embodiments, a pro-inflammatory modulator is selected from: interleukin 1β (IL-1β), IL-6, tumor necrosis factor α (TNF-α), interferon γ (IFN-γ), reactive oxidative species (ROS), IL-8, complement C3, complement C5, or any combination thereof. In some embodiments, a pro-inflammatory modulator is selected from: ROS, IL-8, complement C3, complement C5, or any combination thereof. In some embodiments, treating comprises reducing a pro-inflammatory modulator as disclosed herein by at least 2%, at least 5%, at least 10%, at least 15%, at least 20%, at least 25%, at least 35%, at least 40%, at least 45%, at least 50%, at least 55%, at least 60%, at least 65%, at least 70%, at least 80%, at least 85%, at least 90%, at least 95%, at least 99%, or any combination thereof. Each possibility presents a separate embodiment of the invention. In some embodiments, reducing a pro-inflammatory modulator is compared to a positive control subject, as disclosed herein. In some embodiments, a positive control subject comprises a subject afflicted with AMD, not treated with the pharmaceutical composition disclosed herein.

In some embodiments, administering comprises orally administering, intravenously administering, intramuscularly administering, or any combination thereof.

In some embodiments, the method further comprises administering to the subject an additional therapeutic, e.g., anti-inflammatory agent. In some embodiments, an additional therapeutic is selected from: an anti-inflammatory agent, an immunosuppressant agent, a small molecule, a neutralizing antibody, or any combination thereof. In some embodiments, an anti-inflammatory agent comprises aminosalicylate (e.g., mesalamine, balsalazide or olsalazine), or a corticosteroid. In some embodiments, an immunosuppressant agent comprises azathioprine, mercaptopurine or methotrexate. In some embodiments, a small molecule comprises tofacitinib, upadacitinib, ozanimod or any combination thereof. In some embodiments, a neutralizing antibody comprises infliximab, adalimumab, golimumab, certolizumab, vedolizumab, ustekinumab, risankizumab, or any combination thereof. In some embodiments, an additional therapeutic comprises antibiotics therapy (e.g., ciprofloxacin or metronidazole).

In some embodiments, the expression of a cytokine, a chemokine, or a competent system protein comprises measuring protein product levels. In some embodiments, the expression, or the level of expression, of a protein or a polypeptide of interest can be detected through flow cytometry, immunohistochemical staining of tissue slices or sections. Additionally, proteins/polypeptides of interest may be detected by western blotting, ELISA or Radioimmunoassay (RIA) assays employing protein-specific antibodies. Alternatively, protein levels can be determined by constructing an antibody microarray in which binding sites comprise immobilized, preferably monoclonal, antibodies specific to a plurality of proteins of interest. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array, and their binding is assayed with assays known in the art.

In some embodiments, the expression of a cytokine, a chemokine, or a competent system protein, comprises measuring gene expression levels (e.g., mRNA). Numerous methods are known in the art for measuring expression levels of a one or more gene such as by amplification of nucleic acids (e.g., PCR, isothermal methods, rolling circle methods, etc.) or by quantitative in situ hybridization. Design of primers for amplification of specific genes is well known in the art, and such primers can be found or designed on various websites such as http://bioinfo.ut.ee/primer3-0.4.0/ or https://pga.mgh.harvard.edu/primerbank/for example. The skilled artisan will understand that these methods may be used alone or combined. Non-limiting exemplary method are described herein.

RT-qPCR: A common technology used for measuring RNA abundance is RT-qPCR where reverse transcription (RT) is followed by real-time quantitative PCR (qPCR). Reverse transcription first generates a DNA template from the RNA. This single-stranded template is called cDNA. The cDNA template is then amplified in the quantitative step, during which the fluorescence emitted by labeled hybridization probes or intercalating dyes changes as the DNA amplification process progresses. Quantitative PCR produces a measurement of an increase or decrease in copies of the original RNA and has been used to attempt to define changes of gene expression in cancer tissue as compared to comparable healthy tissues.

RNA-Seq: RNA-Seq uses recently developed deep-sequencing technologies. In general, a population of RNA (total or fractionated, such as poly(A)+) is converted to a library of cDNA fragments with adaptors attached to one or both ends. Each molecule, with or without amplification, is then sequenced in a high-throughput manner to obtain short sequences from one end (single-end sequencing) or both ends (pair-end sequencing). The reads are typically 30-400 bp, depending on the DNA-sequencing technology used. In principle, any high-throughput sequencing technology can be used for RNA-Seq. Following sequencing, the resulting reads are either aligned to a reference genome or reference transcripts, or assembled de novo without the genomic sequence to produce a genome-scale transcription map that consists of both the transcriptional structure and/or level of expression for each gene. To avoid artifacts and biases generated by reverse transcription direct RNA sequencing can also be applied.

Microarray: Expression levels of a gene may be assessed using the microarray technique. In this method, polynucleotide sequences of interest (including cDNAs and oligonucleotides) are arrayed on a substrate. The arrayed sequences are then contacted under conditions suitable for specific hybridization with detectably labeled cDNA generated from RNA of a test sample. As in the RT-PCR method, the source of RNA typically is total RNA isolated from a tumor sample, and optionally from normal tissue of the same patient as an internal control or cell lines. RNA can be extracted, for example, from frozen or archived paraffin-embedded and fixed (e.g., formalin-fixed) tissue samples. For archived, formalin-fixed tissue cDNA-mediated annealing, selection, extension, and ligation, DASL-Illumina method may be used. For a non-limiting example, PCR amplified cDNAs to be assayed are applied to a substrate in a dense array. Microarray analysis can be performed by commercially available equipment, following manufacturer's protocols, such as by using the Affymetrix GenChip technology, or Incyte's microarray technology.

As used herein the terms “reducing” and “decreasing” are used interchangeably to refer to a statistically significant reduction in the expression and/or activity. In one embodiment significant reduction refers to a reduction of at least 10%, or alternatively at least 20%, or alternatively at least 30%, or alternatively at least 40%, or alternatively at least 50%, or alternatively at least 60%, or alternatively at least 70%, or alternatively at least 75%, or alternatively at least 80%, or alternatively at least 85%, or alternatively at least 90%, or alternatively at least 95%, or alternatively at least 97% or alternatively at least 99% reduction in expression and/or activity. Each possibility represents a separate embodiment of the present invention.

The term “therapeutically effective amount” refers to the concentration of the compound that is normalized to body weight (BW) and is effective to treat a disease or disorder in a mammal. The term “a therapeutically effective amount” refers to an amount effective, at dosages and for periods of time necessary, to achieve the desired therapeutic or prophylactic result. A physician of ordinary skill can readily determine and prescribe the effective amount of the bioactive agent required. The exact dosage form and regimen would be determined by the physician according to the patient's condition. The dosage administered will be dependent upon the age, health, and weight of the recipient, kind of concurrent treatment, if any, frequency of treatment, and the nature of the effect desired.

As used herein, the terms “administering,” “administration,” and like terms refer to any method which, in sound medical practice, delivers a composition containing an active agent to a subject in such a manner as to provide a therapeutic effect. One aspect of the present subject matter provides for oral administration of a therapeutically effective amount of a composition of the present subject matter to a patient in need thereof. Other suitable routes of administration can include parenteral, subcutaneous, intravenous, intramuscular, or intraperitoneal. The dosage administered will be dependent upon the age, health, and weight of the recipient, kind of concurrent treatment, if any, frequency of treatment, and the nature of the effect desired.

In some embodiments, the route of administration of the pharmaceutical composition disclosed herein comprises an intravenous route, an intramuscular route, a subcutaneous route, or an oral delivery route. The route of administration of the pharmaceutical composition will depend on the disease or condition to be treated. Suitable routes of administration include, but are not limited to, parenteral injections, e.g., intradermal, intravenous, intramuscular, intralesional, subcutaneous, intrathecal, and any other mode of injection as known in the art. Although the bioavailability of peptides administered by other routes can be lower than when administered via parenteral injection, by using appropriate compositions it is envisaged that it will be possible to administer the compositions of the invention via transdermal, oral, rectal, vaginal, topical, nasal, inhalation and ocular modes of treatment. In addition, it may be desirable to introduce the pharmaceutical compositions of the invention by any suitable route, including intraventricular and intrathecal injection; intraventricular injection may be facilitated by an intraventricular catheter, for example, attached to a reservoir. In some embodiments, the composition of the invention comprising oral delivery. In some embodiments, the composition of the invention comprises an oral composition. In some embodiments, the composition of the invention further comprises orally acceptable carrier, excipient, or a diluent.

As used herein, the terms “subject” or “individual” or “animal” or “patient” or “mammal” refers to any subject, particularly a mammalian subject, for whom therapy is desired, for example, a human.

In some embodiments, the pharmaceutical composition comprises a pharmaceutically acceptable carrier, adjuvant, or excipient.

As used herein, the term “carrier”, “adjuvant”, or “excipient” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

As used herein, the term “about” when combined with a value refers to plus and minus 10% of the reference value. For example, a length of about 1000 nanometers (nm) refers to a length of 1000 nm±100 nm.

It is noted that as used herein and in the appended claims, the singular forms “a”, “an”, and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely”, “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

C57BL mice 8-10 weeks old were given 2.5% DSS in their drinking water for 7 days. On the fourth day, the mice were injected subcutaneously (S.C) with either DMSO or 2-CL-MGV-1 (30 μl) for four days. The mice were sacrificed on the eighth day of the experiment.

### Example 1

### 2-Cl-MGV-1 Inhibits Inflammation In Vivo in Inflammatory Bowel Disease

The present invention relates to 2-chlorophenyl quinazolin-4-yl dimethylcarbamate (2-Cl-MGV-1), a heterocyclic compound based on a quinazoline scaffold, which binds to the mitochondrial translocator protein (TSPO) and was suggested to be effective against traumatic brain injury and brain infection. In the present invention, the inventors demonstrate that 2-Cl-MGV-1 counteracts inflammation, ROS generation and cytokine release, in a peripheral inflammatory and/or degenerative disease (e.g. inflammatory and/or degenerative disease outside the CNS).

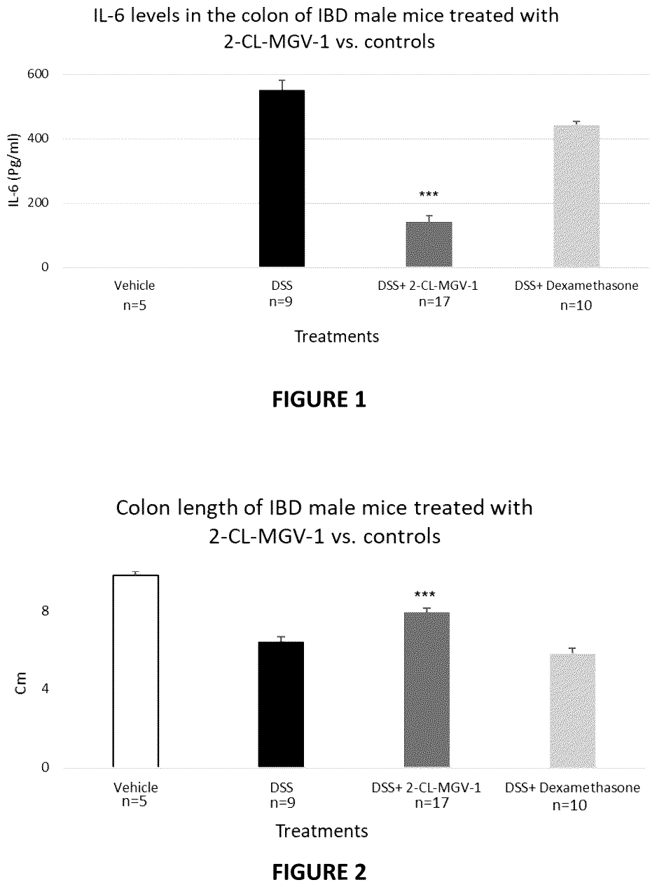

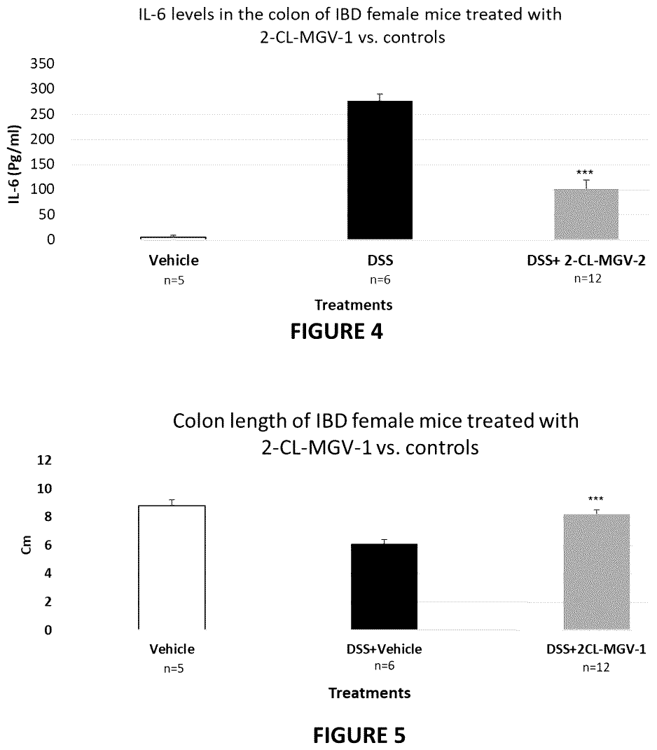

In the IBD experiment, 2-Cl-MGV-1 was injected subcutaneously for four days, three days after inflammation induction in the colon, using dextran sulfate sodium (DSS). On the following day, the mice were sacrificed, and the colon was removed. Colon lengths were measured, and cytokine levels were detected by ELISA. As demonstrated in FIGS. 1 and 4, IL-6 levels within the colon, were increased dramatically following treatment with DSS (which mimicks IBD state). Treatment with 2-CL-MGV-1 after inflammation significantly reduced IL-6 levels in both male and female mice (FIGS. 1 and 4, respectively). Surprisingly, treatment with 2-CL-MGV-1 reduced IL-6 levels by 75% or 64% for male and female mice, respectively, whereas the reduction achieved by treatment with dexamethasone (DEX) was only 20% for male mice (FIG. 1).

Moreover, treatment of mice with DSS typically causes colon shrinkage. As may be seen in FIGS. 2 and 5, 2-CL-MGV-1 treatment alleviated the colon shrinkage effect, in both male and female mice, respectively. As demonstrated for the IL-6 effect, this effect was significantly more pronounced compared to that of treatment with dexamethasone in male mice.

Hence, the inventors are the first to show that 2-CL-MGV-1 has a therapeutic potential in IBD, as shown in both male and female mice. The results also demonstrate that 2-CL-MGV-1 is superior even to dexamethasone, a potent steroid which is a first-line treatment in IBD.

### Example 2

### 2-Cl-MGV-1 Inhibits Inflammation In Vitro in Age-Related Macular Degeneration

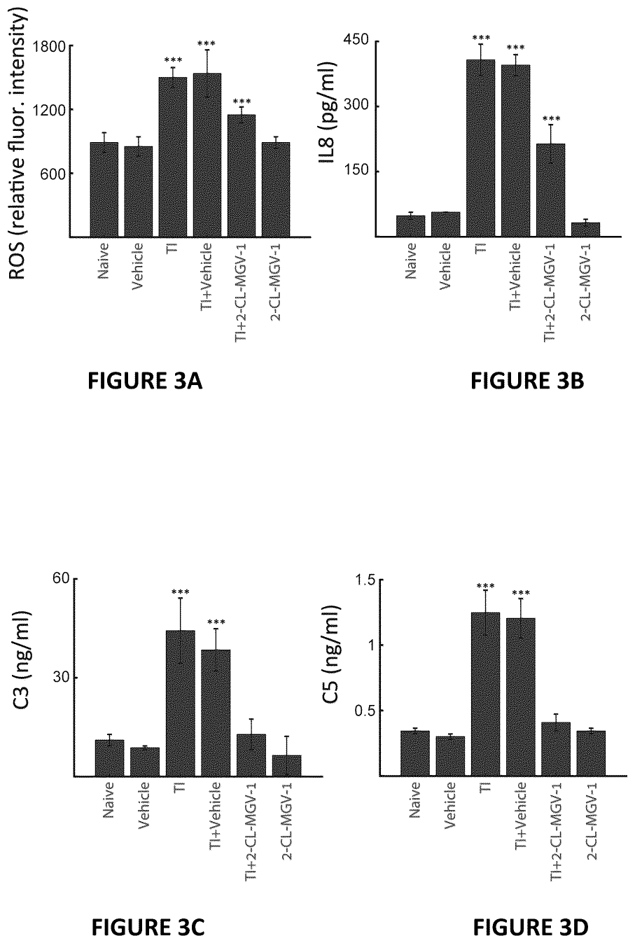

Next, the effect of 2-CL-MGV-1 was examined in an in vitro model of age-related macular degeneration (AMD). Human retinal pigment epithelium (RPE)-19 cell line stimulated with TNF-α and INF-γ (TI) are known to mimic AMD by complement activation and increased levels of circulating cytokines. As demonstrated in FIG. 3, TI-stimulated RPE cells secreted elevated levels of pro-inflammatory mediators, comprising reactive oxygen species (ROS), IL-8, complement C3, and complement C5. Interestingly, pretreatment of RPE cells with the TSPO ligand, 2-Cl-MGV-1, significantly reduced the TI-induced secretion of all the examined pro-inflammatory mediators, indicating that 2-CL-MGV-1 has a therapeutic potential in AMD.

In summary, the inventors demonstrated that the TSPO ligand 2-Cl-MGV-1 is effective in treating and decreasing inflammation in peripheral inflammatory and/or degenerative diseases, comprising IBD and AMD.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1.-7. (canceled)

8. A method for treating or reducing peripheral inflammation, or a symptom associated therewith in a subject in need thereof, wherein said peripheral inflammation is in a site or an organ excluding the central nervous system (CNS) of said subject, the method comprising administering to said subject a therapeutically effective amount of a pharmaceutical composition comprising a compound represented by Formula I: 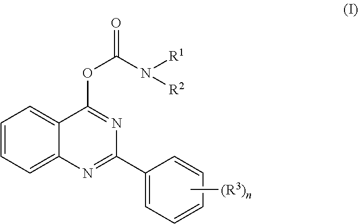
wherein R1 and R2 are each independently a linear or branched C1-C12 alkyl; R3 is a halogen; and n is 1, 2, 3, 4 or 5; including salts thereof, thereby treating or reducing a peripheral inflammation or a symptom associated therewith in the subject.

9. The method of claim 8, wherein said compound represented by Formula I comprises 2-Cl-MGV-1.

10. The method of claim 8, wherein said site or organ is selected from the group consisting of: intestine, eye, lung, heart, and any combination thereof, of said subject.

11. The method of claim 8, wherein said subject is afflicted with a disease selected from the group consisting of: an autoimmune disease, an age-related disease, a pathogen-induced infectious disease, and any combination thereof.

12. The method of claim 11, wherein said autoimmune disease is inflammatory bowel disease (IBD).

13. The method of claim 12, wherein said treating or reducing peripheral inflammation comprises reducing colon shortening in said subject compared to a control subject.

14. The method of claim 11, wherein said age-related disease is age-related macular degeneration (AMD).

15. The method of claim 8, wherein said treating or reducing peripheral inflammation comprises reducing the expression, secretion, or both, of a pro-inflammatory modulator, in said subject compared to a control subject.

16. The method of claim 15, wherein said pro-inflammatory modulator is selected from the group consisting of: interleukin 1β (IL-1β), IL-6, tumor necrosis factor α (TNF-α), interferon γ (IFN-γ), reactive oxidative species (ROS), IL-8, complement C3, complement C5, and any combination thereof.

17. The method of claim 8, wherein said administering comprises orally administering, intravenously administering, intramuscularly administering, or any combination thereof.

18. The method of claim 8, further comprising administering to said subject an anti-inflammatory agent.

19. The method of claim 18, wherein said anti-inflammatory agent comprises a corticosteroid or an aminosalicylate.

20. The method of claim 19, wherein said corticosteroid comprises dexamethasone.

21. The method of claim 19, wherein said aminosalicylate comprises mesalamine, balsalazide or olsalazine.

22. The method of claim 8, wherein said reducing is compared to a control subject.
